(19) United States
(12) Patent Application Publication
(10) Pub. No.: US 2025/0114597 A1
(43) Pub. Date: Apr. 10, 2025

(54) HELMET FOR MENTAL STATE REGULATION

(71) Applicant: Beijing Institute of Technology, Beijing (CN)

(72) Inventors: Bin HU, Beijing (CN); Qunxi DONG, Beijing (CN); Lixian ZHU, Beijing (CN); Fuze TIAN, Beijing (CN); Ran CAI, Beijing (CN); Kun QIAN, Beijing (CN); Mingzhe FEI, Beijing (CN)

(73) Assignee: Beijing Institute of Technology, Beijing (CN)

(21) Appl. No.: 18/898,669

(22) Filed: Sep. 26, 2024

(30) Foreign Application Priority Data
Oct. 7, 2023 (CN) 202311276975.2

(51) Int. Cl. A61N 1/36; A42B 3/04; A61N 1/04

(52) U.S. Cl. CPC A61N 1/36025; A42B 3/0406; A61N 1/0456; A61N 1/0484; A61N 1/36031

(57) ABSTRACT

The provided is a helmet for mental state regulation. The device includes: a helmet, an electroencephalogram (EEG) acquisition module set to the helmet, a signal analysis module, a vagus nerve stimulation module and a vagus nerve stimulation ear clip; the EEG acquisition module is used to acquire an EEG signal of a target user in real time, and preprocess the acquired EEG signal; the signal analysis module is used to extract features of the preprocessed EEG signal, and the extracted EEG features are input into a mental state assessment model to obtain a mental state value of the target user; the vagus nerve stimulation module is used to determine stimulation parameters of the vagus nerve stimulation ear clip according to the mental state value of the target user, and adjust the stimulation parameters adaptively and dynamically by obtaining the EEG features in real time.

### CROSS-REFERENCE TO THE RELATED APPLICATIONS

This application is based upon and claims priority to Chinese Patent Application No. 202311276975.2, filed on Oct. 7, 2023, the entire contents of which are incorporated herein by reference.

### TECHNICAL FIELD

The present invention relates to the field of mental state regulation technology, in particular to a helmet for mental state regulation.

### BACKGROUND

Diseases closely related to mental function can cause abnormal mental states in the early stage, which seriously threatens human mental health. At present, the clinical diagnosis of mental abnormalities such as depression is mainly based on medical history, interview, observation, scale filling, and questionnaire assessment, and the physiological response of patients is collected by multi-channel physiological instrument for auxiliary diagnosis. This detection method relies on the experience and subjective factors of doctors, and is easily disturbed by the surrounding scene environment, also, the inquiry speed is slow with low efficiency, the instrument is costly, and the volume is large. In addition, the intervention methods are mostly drug intervention, with large side effects, easy to repeat, and difficult to assess the intervention effect, and there are often intersections between various abnormal states, resulting in poor assessment and intervention effects. So it is particularly important to develop universal mental state assessment and non-drug intervention techniques. The United States Food and Drug Administration (FDA) officially approved Vagus Nerve Stimulation (VNS) for refractory epilepsy intervention in 1997 and approved it as a complementary alternative therapy for refractory depression in 2005. In recent years, clinical practice has confirmed that acupuncture and moxibustion can effectively improve and intervene in abnormal mental states, its efficacy is good and its adverse reactions are small, which has been included in the latest clinical practice guidelines of the American Society of Internal Medicine.

Although vagus nerve stimulation has a certain effect on the intervention of abnormal mental state, the existing intervention technology lacks effective quantitative assessment methods, has poor robustness, and cannot achieve accurate intervention. Biofeedback technology can carry out an effective quantitative assessment for the intervention of abnormal mental state, but so far, there is no vagus nerve stimulation intervention regulation technology based on biofeedback at domestic and abroad.

### SUMMARY

An objective of the present invention is to provide a helmet for mental state regulation, which improves the effectiveness of vagus nerve stimulation.

To achieve the above objective, the present invention provides the following scheme:
a helmet for mental state regulation, including: a helmet, an electroencephalogram (EEG) acquisition module set to the helmet, a signal analysis module, a vagus nerve stimulation module and a vagus nerve stimulation ear clip;
the EEG acquisition module is configured to acquire an EEG signal of a target user in real-time, and preprocess the acquired EEG signal to obtain a preprocessed EEG signal;
the signal analysis module is configured to extract EEG features of the preprocessed EEG signal, and the extracted EEG features are input into a mental state assessment model to obtain a mental state value of the target user;
the vagus nerve stimulation module is configured to determine stimulation parameters of the vagus nerve stimulation ear clip according to the mental state value of the target user, and adjust the stimulation parameters adaptively and dynamically by obtaining the EEG features in real-time; and
the vagus nerve stimulation ear clip is configured to stimulate a percutaneous ear vagus nerve of the target user.

Optionally, the EEG acquisition module includes a preprocessing unit; the preprocessing unit is configured to filter and amplify the acquired EEG signal in turn to obtain the preprocessed EEG signal.

Optionally, the EEG features include spectral eigenvalues, an Alpha brain wave asymmetry, a Lempel-Ziv Complexity (LZC), and a sample entropy.

Optionally, the stimulation parameters include a stimulation intensity, a stimulation frequency and a stimulation duration, a frequency range is 0 Hz-100 Hz, and a duration range is 0 Min-60 Min.

Optionally, the EEG acquisition module includes an EEG sensor, the EEG sensor is located behind an interior of the helmet.

Optionally, the vagus nerve stimulation module includes a Proportional-Integral-Derivative (PID) control unit based on reinforcement learning, the PID control unit based on the reinforcement learning is configured to input the EEG features obtained in real-time, and the stimulation parameters are adaptively and dynamically adjusted in real-time through the EEG features obtained in real-time.

Optionally, an audio playback module is further included, the audio playback module is configured to output music corresponding to the current real-time acquired EEG signal.

Optionally, the mental state assessment model is established by adopting an extreme Gradient Boosting (XGBoost) model.

According to the specific embodiments provided by the present invention, the present invention discloses the following technical effects:
the present invention determines the stimulation parameters of the vagus nerve stimulation ear clip according to the mental state value of the target user, and adjusts the stimulation parameters adaptively and dynamically by obtaining the EEG features in real-time, and the real-time biofeedback of the mental state, which realizes personalized and precise mental state regulation.

### BRIEF DESCRIPTION OF THE DRAWINGS

To explain the embodiments of the present disclosure or the technical solutions in the prior art more clearly, a brief introduction will be made to the accompanying drawings used in the embodiments. It is obvious that the drawings in the description below are only some embodiments of the present disclosure, and those ordinarily skilled in the art can obtain other drawings according to these drawings without creative work.

FIG. 1 is a schematic diagram of a structure of a helmet for mental state regulation provided by an embodiment of the present invention;

FIG. 2 is a schematic diagram of mental state regulation provided by an embodiment of the present invention;

FIG. 3 is a schematic diagram of a denoising algorithm provided by an embodiment of the present invention;

FIG. 4 is a schematic diagram of a PID control vagus nerve stimulation based on reinforcement learning provided by an embodiment of the present invention;

FIG. 5 is a schematic diagram of an electrode provided by an embodiment of the present invention;

FIG. 6 is a schematic diagram of a workflow of a helmet for mental state regulation provided by an embodiment of the present invention;

FIG. 7 is a schematic diagram of a placement of a master control module provided by an embodiment of the present invention;

FIG. 8 is a schematic diagram of a switch button position provided by an embodiment of the present invention;

FIG. 9 is a schematic diagram of a placement of an audio playback unit and a vagus nerve stimulation ear clip provided by an embodiment of the present invention;

FIG. 10 is a schematic diagram of a flexible electrode position provided by an embodiment of the present invention;

### DETAILED DESCRIPTION OF THE EMBODIMENTS

In the following, the technical solutions in the embodiment of the invention will be described clearly and completely with reference to the attached diagram of the embodiments of the present invention. Apparently, the described embodiments are only some but not all of the embodiments of the present disclosure. All other embodiments obtained by those of ordinary skill in the art based on the embodiments of the present disclosure without involving any creative effort shall fall within the scope of protection of the present disclosure.

An objective of the present invention is to provide a helmet for mental state regulation, which improves the effectiveness of vagus nerve stimulation.

In order that the objectives, features and advantages of the present invention described above may be more obvious and easy to understand, a detailed description of the attached drawings and specific embodiments of the present invention will be set forth in the description which follows.

In view of the existing mental monitoring and non-drug intervention technologies, there are bottlenecks such as low accuracy, poor robustness, scarcity of effective assessment intervention objective indicators, and low universality, the present invention provides a helmet for mental state regulation, its main content is to provide vagus nerve stimulation paradigm and audio stimulation paradigm based on assessment state adaptation through a real-time mental state assessment, and form a closed-loop ‘assessment-regulation-assessment’ biofeedback control of mental state intervention technology. The technology is embedded into the helmets worn daily, and non-disturbing real-time monitoring of the mental state of personnel without changing the basic functions of the original helmet.

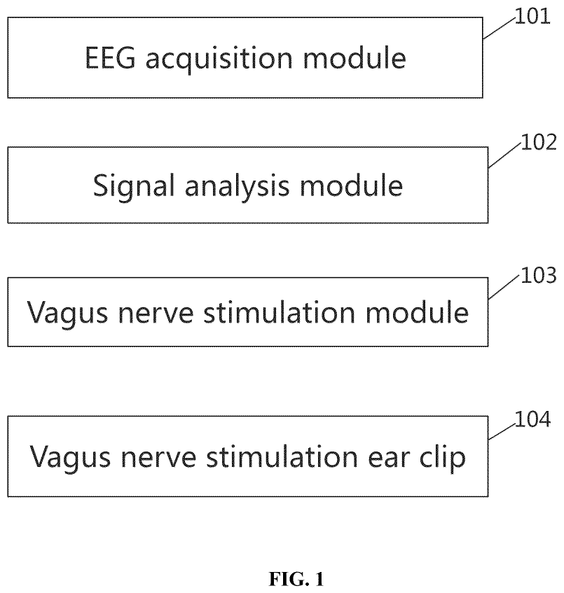

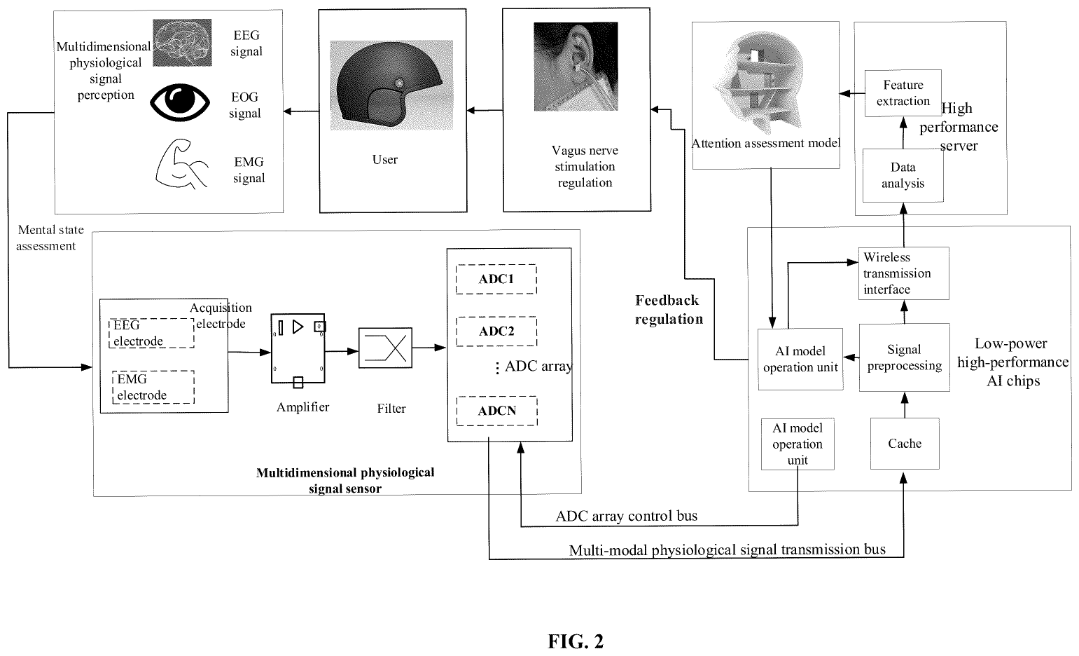

As shown in FIGS. 1-2, the present invention provides a helmet for mental state regulation, including: a helmet (helmet body), an EEG acquisition module set to the helmet (helmet body), a signal analysis module, a vagus nerve stimulation module and a vagus nerve stimulation ear clip;

the EEG acquisition module is used to acquire the EEG signal of a target user in real-time, and preprocess the acquired EEG signal.

The target user is a user wearing the helmet, that is, a user of the helmet of the present invention.

The EEG acquisition module includes a preprocessing unit; the preprocessing unit is used to filter and amplify the acquired EEG signal in turn to achieve filtering and denoising, the preprocessed EEG signal is obtained.

The preprocessing unit includes a 0-45 Hz low-pass FIR filter, and the 0-45 Hz low-pass FIR filter is used to filter the EEG signal. The acquired EEG signal is subject to preprocessing and denoising algorithms such as filtering, amplification, and so on, and finally, through the universal and integrated EEG biological information acquisition module (EEG acquisition module), the continuous monitoring of EEG biological information of the tested patients is realized.

In the preprocessing unit, the adaptive noise removal algorithm based on wavelet decomposition combined with the Kalman filter is adopted, and the specific process is shown in FIG. 3.

The signal analysis module is used to extract features of the preprocessed EEG signal, and the extracted EEG features are input into a mental state assessment model to obtain a mental state value of the target user, and the mental state of the target user is denoted by the mental state value of the target user.

The EEG features include spectral eigenvalues, an Alpha brain wave asymmetry, an LZ complexity, and a sample entropy. The spectral eigenvalues refer to a power spectral density (PSD).

The denoised data is divided into slices with a length of 2000 sample points, each slice extracts relevant feature parameters such as spectral eigenvalues and Alpha asymmetry, among which there are four nonlinear eigenvalues, namely Alpha Power Variation (APV), Lempel-Ziv Complexity, sample entropy, and Frontal Alpha Asymmetry (FAA).

The mental state assessment model is established by adopting an XGBoost model.

The output of the mental state assessment model is the probability value.

In FIG. 2, EEG denotes the EEG signal, electrooculography (EOG) denotes the EOG signal, electromyography (EMG) denotes the EMG signal, and ADC denotes the analog-to-digital converter.

The vagus nerve stimulation module is used to start the stimulation paradigm according to the mental state value of the target user, determine stimulation parameters of the vagus nerve stimulation ear clip, and adjust the stimulation parameters adaptively and dynamically by obtaining the EEG features in real-time.

The vagus nerve stimulation module includes a PID control unit based on reinforcement learning, the PID control unit based on reinforcement learning is used to input the EEG features obtained in real-time, and the stimulation parameters are adaptively and dynamically adjusted in real-time through the EEG features obtained in real-time.

The above-mentioned relevant feature parameters (EEG features) of EEG signal is taken as input, and the probability distribution output by the machine learning model is taken as output, the XGBoost model is established to complete the quantitative assessment of the current mental state, wherein the probability distribution is the probability distribution of EEG signal. The vagus nerve stimulation module triggers the transcutaneous vagus nerve electrical stimulation of the ear, and the stimulation parameters such as the intensity, duration, and frequency of the vagus nerve stimulation will be dynamically and adaptively adjusted by PID according to the quantitative assessment results of the mental state, the feedback regulation output result Y, Y is controlled by the reinforcement learning model, and the reinforcement learning model outputs the frequency, intensity and stimulation duration parameters of the vagus nerve stimulation.

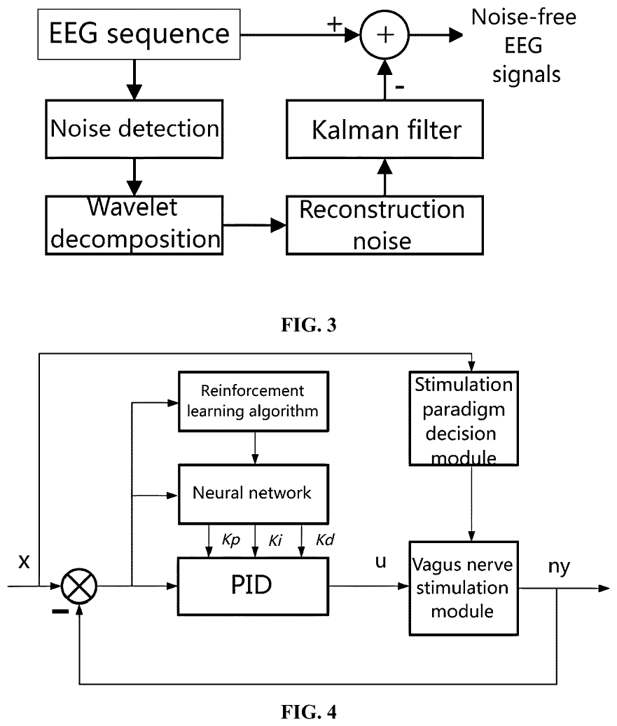

FIG. 4 is a PID control vagus nerve stimulation regulation scheme based on reinforcement learning, firstly, the real-time individual mental state assessment result is extracted as the input x of the reinforcement learning model, and the stimulation paradigm decision module provides the initial vagus nerve stimulation parameters according to the input, meanwhile the EEG features are extracted as feedback, the reinforcement learning module dynamically controls the PID parameters according to the eigenvalues to achieve personalized and accurate regulation. The specific feedback control algorithm flow is:

(1) Calculate the reinforcement learning target parameters:

$${max}_{π ∈ Γ} J ( π ) = {max}_{π ∈ Γ} E [ {\sum }_{k = 1}^{K} {y}^{k - 1} {r}_{k} \vert π ] ;$$

where π denotes a strategy, I denotes an action set, J(π) denotes a long-term optimal value of the state, e denotes an expectation, K denotes a number of states (optimization times), y^{k-1} denotes a discount rate of the k-1^{st} optimization, and rₖ denotes a reward of the k state.

(2) The control signal is generated by the control strategy u, u is satisfied:

$${u}^{*} = arg {max}_{{u}_{k}} Q ( {x}_{k} , {u}_{k} ) ;$$

where u* denotes an optimal value function, and argmaxᵤ_{ₖ} denotes an optimization function.

Where xₖ and uₖ are a state and a control signal of the stimulation system at the k^{th} optimization, and Q(xₖ, uₖ) is an action value function.

$$Q ( {x}_{k} , {u}_{k} ) = E [ {R}_{k} \vert {x}_{k} , {u}_{k} ] ;$$

$${Q}^{*} ( {x}_{k} , {u}_{k} ) = {max}_{μ} E [ {R}_{k} \vert {x}_{k} , {u}_{k} ] ;$$

where Rₖ denotes a reward function, Q*(xₖ, uₖ) denotes an optimal action solved by a recursive method, and u denotes a strategy controller.

(3) The uₖ and Q(xₖ, uₖ) are calculated by the neural network, where the input parameters of the neural network include the parameters of the strategy controller, namely, stimulation intensity, stimulation frequency, and stimulation duration, as well as the reinforcement learning reward parameters and error parameters.

(4) The final network output controls PID parameters Kp, Ki and Kd. Where the speed of error adjustment is controlled by Kp. In addition, it is proportional to the change rate of error, and the change rate of error adjustment is controlled by Kd. The deviation integral is adjusted by Ki, the formula dif(t)=dif(t−1)+Δdif(t) denotes a current vagus nerve intensity level at time t, dif(t−1) denotes a current vagus nerve intensity level at time t−1, and Δdif(t) denotes a difference value between dif(t) and dif(t−1).

The vagus nerve stimulation ear clip is used to stimulate a percutaneous ear vagus nerve of the target user. A vagus nerve stimulation ear clip is placed in the left ears and right ears. The vagus nerve stimulation ear clip is a conductive silica gel material with a resistance value of more than 10KΩ. Paradigm features of vagus nerve stimulation are stimulation parameters.

The stimulation parameters include stimulation intensity, stimulation frequency, and stimulation duration, the frequency range is 0 Hz-100 Hz, the duration range is 0 Min-60 Min, and the stimulation intensity range is 0-100.

In order to realize the ‘continuous’ quantitative perception of mental state, the present invention needs to break through the effective acquisition of biological information and continuous monitoring technology of the general population when wearing helmets, and realize long-term tracking and effective acquisition of biological information in natural situations. The EEG sensor is located behind the interior of the helmet.

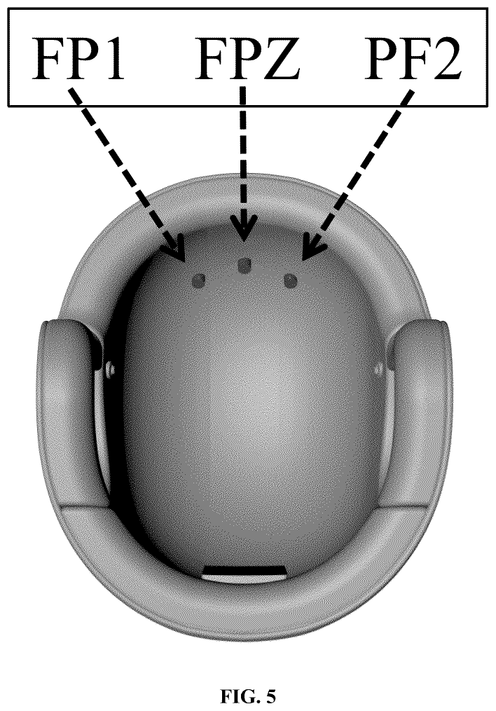

The EEG acquisition module includes an EEG sensor, the EEG sensor is located behind the interior of the helmet, the electrode of the EEG sensor is a contact flexible electrode, the position of the EEG sensor is FP1, FP2, and FPZ in the prefrontal lobe, and the reference electrode is A1 and A2, as shown in FIG. 5.

The helmet for mental state regulation also includes an audio playback module, the audio playback module is used to output corresponding music according to the current real-time acquired EEG signal.

The audio playback module stores different types of mental state regulation music, and the EEG signal of different value ranges correspond to the same type of mental state regulation music. The audio intervention module is used to play different types of mental state regulation music, its working mode can work independently according to the results of mental state assessment, and can also work synchronously with the vagus nerve stimulation module. There are no less than 10 audio types, and the duration of a single play is 0-30 minutes.

The CPU of the helmet control system adopts the RISC-V core brain-like chip, and the EEG signal is transmitted to the chip, the EEG signal is preprocessed by the cut-off frequency 0-45 Hz low-pass FIR filter, and then five features of power spectrum density (PSD), LZ complexity, Alpha asymmetry, sample entropy and Renyi entropy are extracted. Finally, the XGBoost model is used to establish a mental state assessment model. The main control CPU of the closed-loop vagus nerve stimulation module based on biological information feedback adopts low-power STM32. The audio playback module is controlled by the STM32 chip with a built-in replaceable SD memory card.

All modules are embedded inside the helmet and only the switch buttons for each module are placed on the outside of the helmet. The mental state assessment module (signal analysis module), vagus nerve stimulation module and audio playback module of the smart helmet can work independently or simultaneously. The audio playback of the audio stimulation module is located on the left and right sides of the helmet, the interfaces of the EEG acquisition module, the vagus nerve module and the audio playback module are all inside the helmet and do not extend outward.

Through the above intelligent closed-loop ‘assessment-intervention-assessment’ method, the mental state assessment and intervention technology of real-time biofeedback can be applied to real-life scenarios to achieve personalized and accurate mental state assessment and intervention, which provides new theories, methods, and techniques for improving the effectiveness of special personnel, and is an original method that integrates multidisciplinary theories such as information science, brain science, cognitive psychology and medical electronics.

The specific implementation steps of a helmet for mental state regulation are as follows: the user turns on the control system through a switch, the control system includes an EEG acquisition module, a signal analysis module, and a vagus nerve stimulation module, after the initialization of the control system is completed, the EEG acquisition sensor filters and amplifies the EEG signal in real-time, and sends the EEG signal to the physiological calculation dedicated chip, the dedicated chip further performs time-series segmentation of the EEG signal, and evaluates the quality of the EEG signal by fast Fourier transform (FFT). When the EEG signal tends to be stable, the relevant feature parameters such as spectral eigenvalues, Alpha brain wave asymmetry, LZ complexity, and sample entropy are input into the mental state assessment model, the model outputs the mental state value and informs the user through the vibration prompt. According to the assessment results, the user wears the vagus nerve stimulation ear clip and starts the vagus nerve stimulation module. The vagus nerve stimulation module calculates the stimulation intensity (0-100), stimulation frequency (0-100 Hz) and stimulation duration (0-60 min) according to the assessment results, and adaptively and dynamically adjusts the stimulation module parameters by real-time calculation of EEG features. Meanwhile, the audio intervention module can be turned on according to the actual scene, the smart helmet will play music according to the current EEG signal and adjust the music type in real-time according to the EEG signal. The audio playback module and the vagus nerve stimulation module can work independently or simultaneously. Real-time monitoring and regulation of mental state are realized through the above steps.

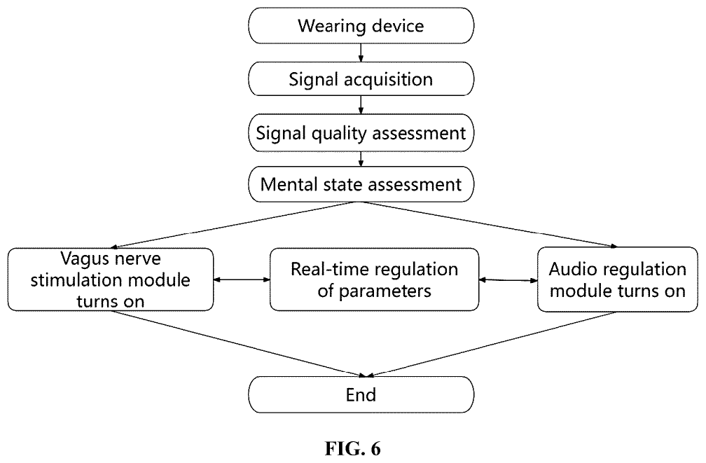

FIG. 6 is the actual operation flow chart, as shown in FIG. 6, the user wears a smart helmet, turns on the device according to the actual application scenario, and acquires EEG signal and frontal lobe body temperature information, the signal quality module evaluates the quality of EEG signal in real-time according to EEG signal for 0-2 minutes. After the signal quality is stable, the mental state assessment module is turned on, the module first acquires 144s resting state EEG signal and then acquires 90s audio stimulation EEG signal, and the mental state assessment module outputs mental state values according to the acquired data. The system turns on the vibration prompt and the ring tone prompt according to the assessment results, the user chooses to turn on the mental state adjustment module according to the prompt, that is, turn on the audio regulation and vagus nerve stimulation module, and wear the vagus nerve stimulation ear clip. After the audio stimulation module is turned on, the system will automatically recommend music according to the current mental state assessment results, and monitor the EEG signal in real-time to adjust the music type, the audio adjustment module works for 30 minutes at a time and can work continuously for 2 hours. Meanwhile, after the vagus nerve stimulation module is turned on, the system will provide the initial vagus nerve stimulation parameters according to the mental state, namely the stimulation frequency, stimulation intensity, and stimulation duration. Then the module will adjust the stimulation paradigm according to the method shown in FIG. 4, and the single adjustment time is 0-60 minutes to achieve refined and personalized mental state regulation.

The audio playback module includes an audio playback control unit and an audio playback unit. The audio playback control unit is used to control the audio playback unit to play music.

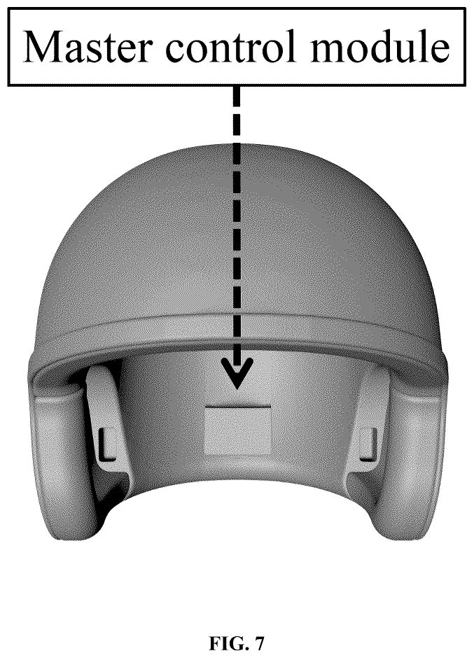

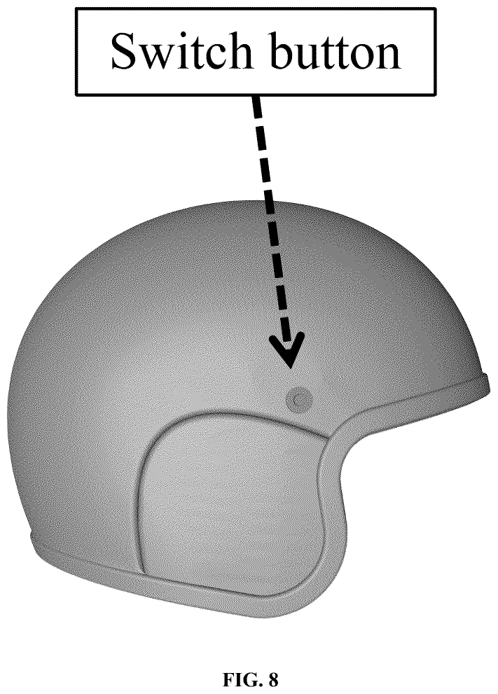

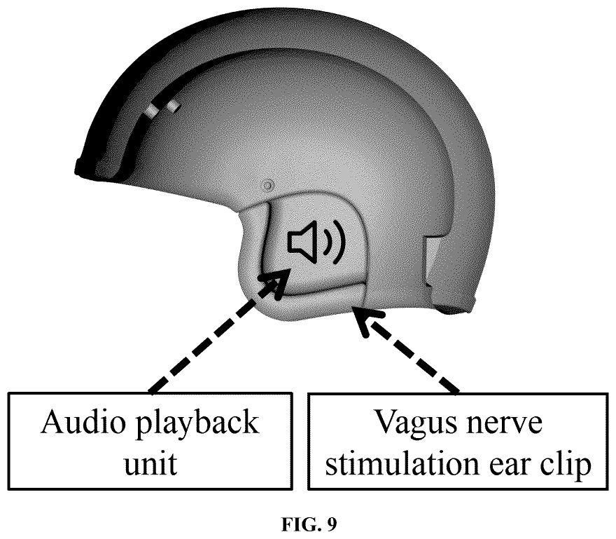

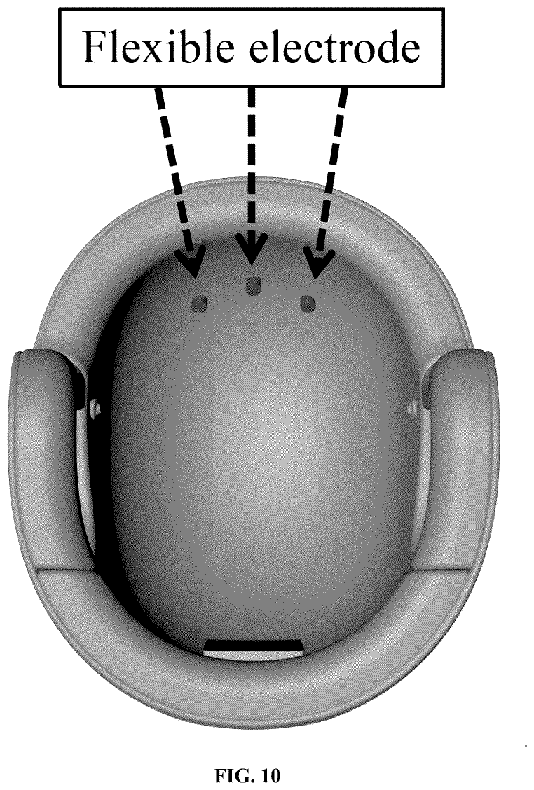

FIGS. 7-10 are the appearance of the smart helmet system and the schematic diagram of its module distribution, wherein FIG. 7 is the schematic diagram of the placement of the master control module (EEG acquisition module, signal analysis module, vagus nerve stimulation module and audio playback control unit), FIG. 9 is the schematic diagram of the placement of the audio playback unit and the vagus nerve stimulation ear clip, the vagus nerve stimulation ear clip is connected by a retractable wire, and the default state is embedded into the ear clip groove dedicated to the helmet. FIG. 10 is a schematic diagram of the flexible electrode, in which the three electrodes FP1, FP2, and FPz in the prefrontal lobe conform to the international 10-20 electrode placement rules. FIG. 8 is a schematic diagram of the switch, in which the switch is a button switch, a single press of 2s is on and off, two consecutive presses are used to adjust the vagus nerve stimulation module, and three consecutive presses are used to adjust the audio playback module.

Each embodiment in this specification is described progressively. Each embodiment focuses on the differences from other embodiments. The same similar parts between each embodiment can be seen in each other.

In this paper, a specific example is used to explain the principle and implementation of the present invention. The above embodiments are only used to help understand the device and its core idea of the present invention. Meanwhile, for the general technical personnel in this field, according to the idea of the present invention, there will be changes in the detailed description of the embodiments and application scope. In summary, the content of this specification should not be understood as a limitation to the present invention.

What is claimed is:

1. A helmet for mental state regulation, comprising: a helmet, an electroencephalogram (EEG) acquisition module set to the helmet, a signal analysis module, a vagus nerve stimulation module and a vagus nerve stimulation ear clip, wherein
the EEG acquisition module is configured to acquire an EEG signal of a target user in real-time, and preprocess the EEG signal to obtain a preprocessed EEG signal;
the signal analysis module is configured to extract EEG features of the preprocessed EEG signal, and the EEG features are input into a mental state assessment model to obtain a mental state value of the target user;
the vagus nerve stimulation module is configured to determine stimulation parameters of the vagus nerve stimulation ear clip according to the mental state value of the target user, and adjust the stimulation parameters adaptively and dynamically by obtaining the EEG features in real-time; and
the vagus nerve stimulation ear clip is configured to stimulate a percutaneous ear vagus nerve of the target user.

2. The helmet for the mental state regulation according to claim 1, wherein the EEG acquisition module comprises a preprocessing unit; the preprocessing unit is configured to filter and amplify the EEG signal in turn to obtain the preprocessed EEG signal.

3. The helmet for the mental state regulation according to claim 1, wherein the EEG features comprise spectral eigenvalues, an Alpha brain wave asymmetry, a Lempel-Ziv Complexity (LZC), and a sample entropy.

4. The helmet for the mental state regulation according to claim 1, wherein the stimulation parameters comprise a stimulation intensity, a stimulation frequency and a stimulation duration, a frequency range is 0 Hz-100 Hz, and a duration range is 0 Min-60 Min.

5. The helmet for the mental state regulation according to claim 1, wherein the EEG acquisition module comprises an EEG sensor, the EEG sensor is located behind an interior of the helmet.

6. The helmet for the mental state regulation according to claim 1, wherein the vagus nerve stimulation module comprises a Proportional-Integral-Derivative (PID) control unit based on reinforcement learning, the PID control unit based on the reinforcement learning is configured to input the EEG features obtained in real-time, and the stimulation parameters are adaptively and dynamically adjusted in real-time through the EEG features obtained in real-time.

7. The helmet for the mental state regulation according to claim 1, wherein an audio playback module is further comprised, the audio playback module is configured to output music corresponding to the current real-time acquired EEG signal.

8. The helmet for the mental state regulation according to claim 1, wherein the mental state assessment model is established by adopting an extreme Gradient Boosting (XGBoost) model.
